(19) United States
(12) Patent Application Publication
(10) Pub. No.: US 2008/0200721 A1
(43) Pub. Date: Aug. 21, 2008

(54) Mdi Production By Means of Liquid Phase and Gas Phase Phosgenation

(75) Inventors: Christian Muller, Mannheim (DE); Eckhard Stroefer, Mannheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/908,363

(22) PCT Filed: Mar. 22, 2006
(86) PCT No.: PCT/EP06/60940

§ 371 Date: Sep. 11, 2007

(30) Foreign Application Priority Data
Mar. 30, 2005 (DE) 10 2005 014 846.8

(51) Int. Cl. C07C 263/10

(52) U.S. Cl. 560/347

(57) ABSTRACT

The invention relates to a process for preparing isocyanates, which comprises the
(1) preparation of a crude MDA mixture by reaction of aniline with formaldehyde,
(2) separation of the crude MDA mixture into MMDA and PMDA,
(3a) phosgenation of the PMDA separated off in step 2 in the liquid phase to form PMDI and
(3b) phosgenation of the MMDA separated off in step 2 in the gas phase to form MMDI.

The invention relates to a process for preparing isocyanates, which comprises the
(1) preparation of a crude MDA mixture by reaction of aniline with formaldehyde,
(2) separation of the crude MDA mixture into MMDA and PMDA,
(3a) phosgenation of the PMDA separated off in step 2 in the liquid phase to form PMDI and
(3b) phosgenation of the MMDA separated off in step 2 in the gas phase to form MMDI.

Aromatic isocyanates are important and versatile raw materials for polyurethane chemistry. MDI in particular is one of the most important industrial isocyanates. In the technical field and for the purposes of the present patent application, the general term “MDI” is used as generic term for methylenedi(phenyl isocyanates) and polymethylene-polyphenylene polyisocyanates. The term methylenedi(phenyl isocyanate) comprises the isomers 2,2′-methylenedi(phenyl isocyanate) (2,2′-MDI), 2,4′-methylenedi(phenyl isocyanate) (2,4′-MDI) and 4,4′-methylenedi(phenylisocyanate) (4,4′-MDI). These isomers are, in the specialist field and for the purposes of the present invention, referred to collectively as “monomeric MDI” or “MMDI”. The term polymethylene-polyphenylene polyisocyanates comprises, in the technical field and for the purposes of the present invention, “polymeric MDI” or “PMDI” comprising higher homologues of monomeric MDI and optionally further comprising monomeric MDI.

In customary industrially relevant production processes, MDI is produced by phosgenation of methylenedi(phenylamine) (MDA). The synthesis occurs in a two-stage process. Firstly, aniline is condensed with formaldehyde to form a mixture of monomeric methylenedi(phenylamines), in the specialist field and for the purposes of the present invention referred to as “MMDA”, and polymethylene-polyphenylene polyamines, in the specialist field and for the purposes of the present invention referred to as “PMDA”, known as crude MDA. The crude MDA usually produced by means of processes of the prior art comprises about 70% of MMDA and is preferably produced at an amine to formaldehyde ratio of from about 2.0 to 2.5.

This crude MDA is subsequently reacted with phosgene in a manner known per se in a second step to give a mixture of the corresponding oligomeric and isomeric methylenedi(phenyl isocyanates) and polymethylene-polyphenylene polyisocyanates, known as crude MDI. Here, the isomer and oligomer composition generally remains unchanged. Part of the 2-ring compounds is then usually separated off in a further process step (e.g. by distillation or crystallization), leaving polymeric MDI (PMDI) as residue.

The phosgenation of the crude MDA mixture is known to those skilled in the art and is described, for example, in “Chemistry and Technology of Isocyanates” by H. Ulrich, John Wiley Verlag, 1996, and in the references cited therein. However, the processes for preparing crude MDI known hitherto from the prior art have numerous disadvantages. Firstly, the space-time yield is undesirably low, for example because of intermediates which precipitate in solid form and react slowly during the preparation, and, secondly, the phosgene holdup in the production plants is undesirably high and the energy requirement for the process is also undesirably high.

It was an object of the invention to provide a process for preparing isocyanates which gives a better space-time yield than the process known from the prior art. Furthermore, a process which makes a lower phosgene holdup in the production plant possible should be provided. In addition, a process which allows a smaller reactor volume in the phosgenation should be provided. Finally, a process which is advantageous from an energy point of view should be provided.

In particular, it was an object of the invention to provide a process having the above advantages for the preparation of MMDI and PMDI. The product mix of MMDI and PMDI in this process should preferably remain essentially unchanged compared to the processes known from the prior art. For the present purposes, the term product mix refers to the composition and amount of PMDI and MMDI produced.

The object has unexpectedly been able to be achieved by separating an aromatic polyamine mixture as is obtained in the methylenedianiline (MDA) process into a fraction of 2-ring MDA isomers (MMDA) and a fraction of MDA isomers having a larger number of rings (PMDA) and subsequently phosgenating these separately, with the phosgenation of the MMDA occurring in the gas phase and the phosgenation of the PMDA occurring in the liquid phase.

The invention accordingly provides a process for preparing isocyanates, which comprises the steps
(1) preparation of a crude MDA mixture by reaction of aniline with formaldehyde,
(2) separation of the crude MDA mixture into MMDA (fraction I) and PMDA (fraction II),
(3a) phosgenation of the PMDA separated off in step (2) in the liquid phase to form PMDI and
   (3b) phosgenation of the MMDA separated off in step (2) in the gas phase to form MMDI.

To carry out the reaction of aniline with formaldehyde to form monomeric methylenedi(phenylamines) (for the purposes of the present invention referred to as “MMDA”) and polymethylene-polyphenylene polyamines (for the purposes of the present invention referred to as “PMDA”), with this mixture of methylenedi(phenylamines) and polymethylene-polyphenylene polyamines being referred to as “crude MDA”, described in step (1), the starting materials are usually mixed in a suitable mixing apparatus. Suitable mixing apparatuses are, for example, mixing pumps, nozzles or static mixers. The starting materials are then reacted in a suitable reaction apparatus, for example in tube reactors, stirred reactors and reaction columns or combinations thereof. The reaction temperature is generally in the range from 20 to 200° C., preferably from 30 to 140° C.

The reaction of step (1) is carried out in the presence of an acid as catalyst, with the catalyst preferably being added in admixture with aniline. Preferred catalysts are mineral acids such as hydrochloric acid, sulfuric acid and phosphoric acid. It is likewise possible to use mixtures of acids. Hydrochloric acid is particularly preferred. If hydrogen chloride is used as catalyst, this can also be used in gaseous form, The amount of catalyst is preferably selected so that a molar ratio of acid/aniline (A/A) of from 0.05 to 0.5, particularly preferably from 0.08 to 0.3, is obtained.

In a preferred embodiment, the reaction of step (1) is carried out in aqueous medium using HCl as catalyst. The reaction can also be carried out in the presence of a solvent. Particularly suitable solvents are ethers, water and mixtures thereof. Examples are dimethylformamide (DMF), tetrahydrofuran (THF) and diethyl isophthalate (DEIP).

Formaldehyde can be supplied to the process of the invention in the form of monomeric formaldehyde and/or in the form of higher homologues, known as poly(oxymethylene)glycols.

The composition of the polyamine mixture produced (crude MDA) is decisively influenced not only by the acid concentration and the temperature but also by the molar ratio of aniline molecules introduced to formaldehyde molecules introduced (A/F ratio) both in the continuous MDA process and the discontinuous MDA process. The greater the A/F ratio selected, the greater the MMDA content of the resulting crude MDA solution. It should be noted in this context that a larger A/F ratio not only leads to a larger proportion of 2-ring molecules (MMDA) but also results in the entire oligomer spectrum of polyamines being shifted in the direction of smaller molecules. For example, the 4-ring MDA content drops by about 80% when the A/F ratio is increased from 2.4 to 5.9.

The molar ratio of aniline:formaldehyde is, for the purposes of the present invention, generally 1.8-10:1, preferably 2-6:1, more preferably 2.1-5.5:1, in particular 2.2-5:1.

The reaction of aniline with formaldehyde can be carried out either continuously or discontinuously, in a batch or semibatch process.

The crude MDA obtained is separated in step (2) of the process of the invention.

The separation of the crude MDA in step (2) can be carried out using the customary methods known from the prior art. The separation is preferably effected by distillation. In a preferred embodiment, the separation is carried out by means of two rectification columns in which aniline is obtained as overhead product in the first column and MMDA is obtained as overhead product in the second column and PMDA is obtained as bottom product in the second column.

In an alternative preferred embodiment, the separation of the amine mixture is carried out in a dividing wall column, in which case the mixture is preferably separated into the following three fractions:

aniline (overhead product), MMDA (product taken off at a side offtake) and PMDA (bottom product).

Step (1) of the process of the invention particularly preferably gives a crude MDA which comprises such small amounts of PMDA that the amine workup can be carried out in one apparatus, e.g. one rectification column, to give the two fractions aniline (overhead product) and MMDA (bottom product).

The purity (in respect of PMDA content) of the MMDA mixture separated off in step (2) (fraction I) should be chosen so that the MMDA mixture (fraction I) can be converted into the gas phase.

For the present purposes, “able to be converted into the gas phase” means that the resulting crude MDA can be transformed from the liquid state into the gaseous state under the action of reaction conditions suitable for the phosgenation, in particular pressure and temperature and, if appropriate, ratio of amine mixture to inert medium or phosgene described below under the process step (3b).

Preference is given to the MMDA separated off in step (2) being able to be converted completely into the gas phase. For the present purposes, “completely” means that not more than 2% by weight, preferably not more than 1% by weight, in particular not more than 0.1% by weight, of a residue which cannot be converted into the gas phase remains.

In a preferred embodiment, the separation of the crude MDA mixture in step (2) is carried out so that the MMDA separated off (fraction I) comprises a PMDA content of from 0 to <12 percent by weight (% by weight), more preferably from 0.1 to <6% by weight, particularly preferably from 0.5 to <3.5% by weight, based on the total weight of MMDA and PMDA.

The purity (in respect of MMDA content) of the PMDA mixture separated off in step (2) (fraction II) is not critical, since the PMDA mixture does not have to be brought into the gas phase. The purity (in respect of MMDA content) of the PMDA mixture separated off in step (2) (fraction II) can be selected according to economic factors.

In a preferred embodiment, the separation of the crude MDA mixture in step (2) is carried out so that the PMDA separated off (fraction II) has an MMDA content of from 0 to <50 percent by weight (% by weight), more preferably from 0.5 to <30% by weight, particularly preferably from 1 to <20% by weight, in particular from 2 to <10% by weight, based on the total weight of PMDA and MMDA.

The separation of the crude MDA mixture in step (2) results in two fractions, firstly a fraction comprising essentially PMDA (fraction II) and a fraction comprising essentially MMDA (fraction I). Fraction (II) is then phosgenated (i.e. a reaction of the amine groups with phosgene to form isocyanate groups occurs) in the liquid phase in process step (3a) and fraction (I) is phosgenated in the gas phase in process step (3b).

The phosgenations carried out separately from one another can be carried out in one plant or in various plants. If they are carried out in various plants, these can also be located at different sites.

The following applies to the liquid-phase phosgenation (3a):

The preparation of the isocyanates is usually carried out by reaction of the corresponding primary amines from fraction (a) with phosgene, preferably an excess of phosgene. This process takes place in the liquid phase. For the purposes of the present invention, “reaction in the liquid phase” means that at least one of the starting material streams is present in the liquid state in the reaction.

An additional inert solvent can be used in the process of the invention. This additional inert solvent is usually an organic solvent or a mixture thereof. Preference is given to chlorobenzene, dichlorobenzene, trichlorobenzene, toluene, hexane, diethyl isophthalate (DEIP), tetrahydrofuran (THF), dimethylformamide (DMF), benzene and mixtures thereof. A particularly preferred solvent is chlorobenzene.

The amine content based on the mixture of amine/solvent is usually in the range from 1 to 50% by mass, preferably from 2 to 40% by mass, particularly preferably from 3 to 30% by mass.

The reaction of step (3a) can be carried out in the customary reactors known from the prior art. It is preferably carried out in a tube reactor.

The tube reactor is preferably heated either via its outer wall or by means of heating elements, e.g. heating coils, or heating tubes comprised in the tube reactor. To narrow the residence time distribution, the tube reactor can be segmented by means of perforated plates. In a further preferred embodiment, the tube reactor has a length (L) to diameter (D) ratio of L/D>6, preferably L/D>10. To build production plants having a high plant capacity, it is also possible to connect a plurality of reactor tubes in parallel.

In step (3a) of the process of the invention, the mixing of the reactants is preferably carried out in a mixing apparatus in which the reaction stream passed through the mixing apparatus is subjected to high shear. Preference is given to using a rotary mixing apparatus, mixing pump or a mixing nozzle located upstream of the reactor as mixing apparatus. Particular preference is given to using a mixing nozzle. The mixing time in this mixing apparatus is usually from 0.0001 s to 5 s, preferably from 0.0005 to 4 s, particularly preferably from 0.001 s to 3 s. For the purposes of the invention, the mixing time is the time which elapses from the beginning of the mixing process until 97.5% of the fluid elements of the resulting mixture have a mixture fraction which, based on the theoretical final value of the mixture fraction of the resulting mixture on reaching the state of perfect mixing, deviates by less than 2.5% from this final value of the mixture fraction (for the concept of the mixture fraction, see, for example, J. Warnatz, U. Maas, R. W. Dibble: Verbrennung, Springer Verlag, Berlin Heidelberg New York, 1997, 2nd edition, p. 134).

In a preferred embodiment, the reaction of amine with phosgene is carried out at absolute pressures of from 0.9 bar to 400 bar, preferably from 3 to 35 bar. The molar ratio of phosgene to amino groups in the feed is generally from 1.1:1 to 12:1, preferably from 1.25:1 to 8:1. The total residence time in the reactors is generally from 10 seconds to 15 hours, preferably from 3 minutes to 12 hours. The reaction temperature is generally from 25 to 260° C. (degrees celsius), preferably from 35 to 240° C.

Step (3a) of the process of the invention is preferably carried out in a single stage. For the purposes of the present invention, this means that mixing and reaction of the starting materials is carried out in one step in a temperature range from 60 to 200° C.. In contrast thereto, many processes known from the prior art are carried out in two stages, i.e. mixing of the starting materials occurs at about 30° C. (resulting in formation of carbamoyl chloride; this step is often referred to as cold phosgenation) and the mixed starting materials are subsequently heated at from about 120 to 200° C. (resulting in dissociation of the carbamoyl chloride to form isocyanate; this stage is often referred to as hot phosgenation).

Step (3a) of the process of the invention can be carried out continuously, semicontinuously, or batchwise. It is preferably carried out continuously.

After the reaction, the mixture is preferably separated by means of rectification into isocyanate(s), solvent, phosgene and hydrogen chloride. Small amounts of by-products remaining in the isocyanate can be separated from the desired isocyanate by means of additional rectification or else crystallization.

Depending on the choice of reaction conditions, the product can comprise inert solvent, carbamoyl chloride and/or phosgene and can be processed further by known methods (cf., for example, WO 99/40059).

The following applies to the gas-phase phosgenation (3b):

The preparation of the isocyanates is usually carried out by reaction of the corresponding primary amines from fraction (b) with phosgene, preferably an excess of phosgene. This process takes place in the gas phase. For the purposes of the present invention, “reaction in the gas phase” means that the starting material streams react with one another in the gaseous state.

The reaction of phosgene with amine fraction (b) occurs in a reaction space which is generally located in a reactor, i.e. the reaction space is the space in which the reaction of the starting materials occurs, while the reactor is the technical apparatus which comprises the reaction space. Here, the reaction space can be any customary reaction space which is known from the prior art and is suitable for noncatalytic, single-phase gas reaction, preferably for continuous noncatalytic, single-phase gas reaction, and will withstand the moderate pressures required. Suitable materials for contact with the reaction mixture are, for example, metals such as steel, tantalum, silver or copper, glass, ceramic, enamels or homogeneous or heterogeneous mixtures thereof. Preference is given to using steel reactors. The walls of the reactor can be smooth or profiled. Suitable profiles are, for example, grooves or corrugations.

It is generally possible to use the reactor types known from the prior art. Preference is given to using tube reactors.

In the process of the invention, the mixing of the reactants occurs in a mixing apparatus in which the reaction stream passed through the mixing apparatus is subjected to high shear. Preference is given to using a static mixing apparatus or a mixing nozzle located upstream of the reactor as mixing apparatus. Particular preference is given to using a mixing nozzle.

The reaction of phosgene with amine in the reaction space usually occurs at absolute pressures of from >1 bar to <50 bar, preferably from >2 bar to <20 bar, more preferably from 3 bar to 15 bar, particularly preferably from 3.5 bar to 12 bar, in particular from 4 to 10 bar.

in general, the pressure in the feed lines to the mixing apparatus is higher than the pressure in the reactor indicated above, Depending on the choice of mixing apparatus, this pressure drops. The pressure in the feed lines is preferably from 20 to 1000 mbar, particularly preferably from 30 to 200 mbar, higher than in the reaction space.

The pressure in the work-up apparatus is generally lower than in the reaction space. The pressure is preferably from 50 to 500 mbar, particularly preferably from 80 to 150 mbar, lower than in the reaction space.

Step (3b) of the process of the invention can, if appropriate, be carried out in the presence of an additional inert medium. The inert medium is a medium which is present in gaseous form in the reaction space at the reaction temperature and does not react with the starting materials at this temperature. The inert medium is generally mixed with amine and/or phosgene prior to the reaction. For example, it is possible to use nitrogen, noble gases such as helium or argon or aromatics such as chlorobenzene, dichlorobenzene or xylene. Preference is given to using nitrogen as inert medium. Particular preference is given to monochlorobenzene or a mixture of monochlorobenzene and nitrogen.

The inert medium is generally used in such an amount that the molar ratio of inert medium to amine is from >2 to 30, preferably from 2.5 to 15. The inert medium is preferably introduced into the reaction space together with the amine.

In the process of the invention, the temperature in the reaction space is selected so that it is below the boiling point of the highest-boiling amine used, based on the pressure conditions prevailing in the reaction space. Depending on the amine (mixture) used and the pressure set, an advantageous temperature in the reaction space is usually from >200° C. to <600° C., preferably from 280° C. to 400° C.

To carry out step (3b), it can be advantageous to preheat the streams of reactants prior to mixing, usually to temperatures of from 100 to 600° C., preferably from 200 to 400° C.

The mean contact time of the reaction mixture in step (3b) of the process of the invention is generally from 0.1 second to <5 seconds, preferably from >0.5 second to <3 seconds, particularly preferably from >0.6 second to <1.5 seconds. For the purposes of the present invention, the mean contact time is the period of time from the commencement of mixing the starting materials until they leave the reaction space. In a preferred embodiment, the dimensions of the reaction space and the flow velocities are selected so that turbulent flow, i.e. flow at a Reynolds number of at least 2300, preferably at least 2700, occurs, with the Reynolds number being calculated using the hydraulic diameter of the reaction space. The gaseous reactants preferably pass through the reaction space at a flow velocity of from 3 to 180 meters/second, preferably from 10 to 100 meters/second.

In the process of the invention, the molar ratio of phosgene to amino groups in the feed is usually from 1:1 to 15:1, preferably from 1.2:1 to 10:1, particularly preferably from 1.5:1 to 6:1.

In a preferred embodiment, the reaction conditions are selected so that the reaction gas at the outlet from the reaction space has a phosgene concentration of more than 25 mol/m³, preferably from 30 to 50 mol/m³. Furthermore, the inert medium concentration at the outlet from the reaction space is generally more than 25 mol/m³, preferably from 30 to 100 mol/m³.

In a particularly preferred embodiment, the reaction conditions are selected so that the reaction gas at the outlet from the reaction space has a phosgene concentration of more than 25 mol/m³, in particular from 30 to 50 mol/m³, and at the same time has an inert medium concentration of more than 25 mol/m³, in particular from 30 to 100 mol/m³.

The reaction volume is usually heated via its exterior surface. To build production plants having a high plant capacity, a plurality of reactor tubes can be connected in parallel.

The process of the invention is preferably carried out in a single stage. For the purposes of the invention, this means that the mixing and reaction of the starting materials occurs in one step and in one temperature range, preferably in the abovementioned temperature range. Furthermore, the process of the invention is preferably carried out continuously.

After the reaction, the gaseous reaction mixture is generally scrubbed with a solvent, preferably at temperatures above 150° C.. Preferred solvents are hydrocarbons which are optionally substituted with halogen atoms, for example chlorobenzene, dichlorobenzene, and toluene. Particular preference is given to using monochlorobenzene as solvent. In the scrub, the isocyanate is selectively transferred into the scrub solution. The remaining gas and the scrub solution obtained are subsequently separated into isocyanate(s), solvent, phosgene and hydrogen chloride, preferably by means of rectifiction. Small amounts of by-products remaining in the isocyanate (mixture) can be separated from the desired isocyanate (mixture) by means of additional rectification or else crystallization.

It is in principle possible to mix the product streams of MMDI and PMDI (completely or partly) again after the phosgenation in steps (3a) and (3b). This can occur after or before the work-up. If mixing is carried out prior to the work-up, the mixed streams can be worked up jointly.

Preference is given to working up the product streams of MMDI and PMDI separately.

After separate work-up, the products MMDI and PMDI can be mixed (completely or partly) and sold as a mixture and/or they can be sold as individual products.

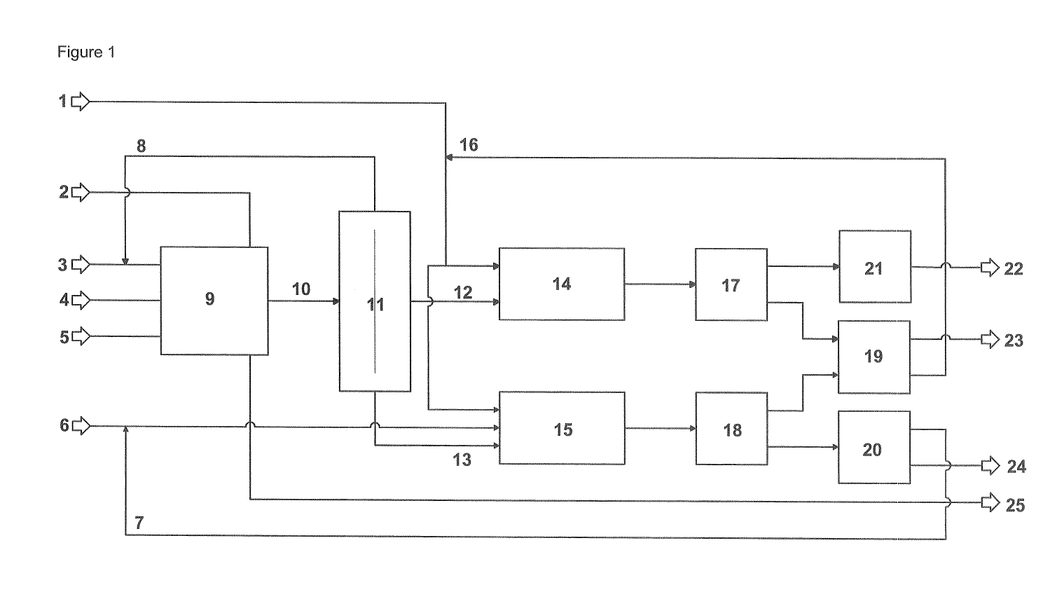

A preferred embodiment of the process of the invention is depicted in FIG. 1.

In FIG. 1:
1 Phosgene
2 Base
3 Aniline
4 Formaldehyde
5 Hydrochloric acid
6 Solvent
7 Recirculated solvent
8 Recirculated aniline
9 MDA reaction space
10 Aniline, MMDA, PMDA
11 Amine separation
12 MMDA
13 PMDA
14 Reaction space for gas-phase phosgenation
15 Reaction space for liquid-phase phosgenation
16 Recirculated phosgene
17 Separation of MMDI/solvent from HCl/phosgene
18 Separation of PMDI/solvent from HCl/phosgene
19 Separation of HCl from phosgene
20 Separation of PMDI from solvent
21 Separation of MMDI from solvent
22 MMDI
23 HCl
24 PMDI
29 Aqueous salt solution (e.g. NaCl, when using HCl and NaOH as base)

1-4. (canceled)

5. A process for preparing isocyanates, wherein said process comprises:
(1) preparing a crude MDA mixture by reacting aniline with formaldehyde;
(2) separating said crude MDA mixture into a gas phase comprising MMDA and a liquid phase comprising PMDA;
(3a) phosgenating said PMDA in said liquid phase separated off in (2) to form PMDI; and
(3b) phosgenating said MMDA in said gas phase separated off in (2) to form MMDI.

6. The process for preparing isocyanates according to claim 5, wherein said reacting of aniline with formaldehyde in (1) is carried out at a ratio of aniline to formaldehyde ranging from 2 to 5.5.

7. The process for preparing isocyanates according to claim 5, wherein said separating of said crude MDA mixture in (2) is carried out so that said MMDA separated off comprises a maximum content of PMDA of 12 wt. %.

8. The process for preparing isocyanates according to claim 5, wherein said separating of said crude MDA mixture in (2) is carried out so that said PMDA separated off comprises a maximum content of MMDA of 30 wt. %.
